(19) United States
(12) Patent Application Publication
(10) Pub. No.: US 2006/0100145 A1
(43) Pub. Date: May 11, 2006

(54) Human chondroosteomodulin (tig2), production, and use for the treatment or diagnosis of bone diseases, cartilage diseases, cartilage diseases, obesity, inflammatory diseases, and skin diseases

(75) Inventors: Wolfgang Meder, Hannover (DE); Martin Wendland, Hannover (DE); Harald John, Hannover (DE); Rudolf Richter, Hannover (DE); Markus Meyer, Hannover (DE); Wolf-Georg Forssmann, Hannover (DE)

Correspondence Address: STERNE, KESSLER, GOLDSTEIN & FOX PLLC, 1100 NEW YORK AVENUE, N.W., WASHINGTON, DC, 20005, US

(73) Assignee: IPF Pharmaceuticals GmbH, Hannover (DE)

(21) Appl. No.: 10/533,300

(22) PCT Filed: Oct. 24, 2003
(86) PCT No.: PCT/EP03/11799

§ 371 Date: Oct. 24, 2005

(30) Foreign Application Priority Data
Oct. 31, 2002 (DE) 102 51 205.1

(51) Int. Cl. A61K 38/22; C40B 40/10; C07K 14/575

(52) U.S. Cl. 514/12; 435/7.1; 530/324

(57) ABSTRACT

The invention relates to the polypeptide COM and its derivatives, and methods for its preparation and recovery in a pure or partially purified form from body fluids and tissues, and its use as a medicament.

The invention relates to the polypeptide HF-chondroosteomodulin (COM) and its derivatives, and methods for its preparation and recovery in a pure or partially purified form from body fluids and tissues or by chemical or biotechnological synthesis.

It is the object of the invention to provide further or improved active substances for the treatment or diagnosis of bone and cartilage diseases, obesity as well as inflammatory diseases and skin diseases.

FIGURES

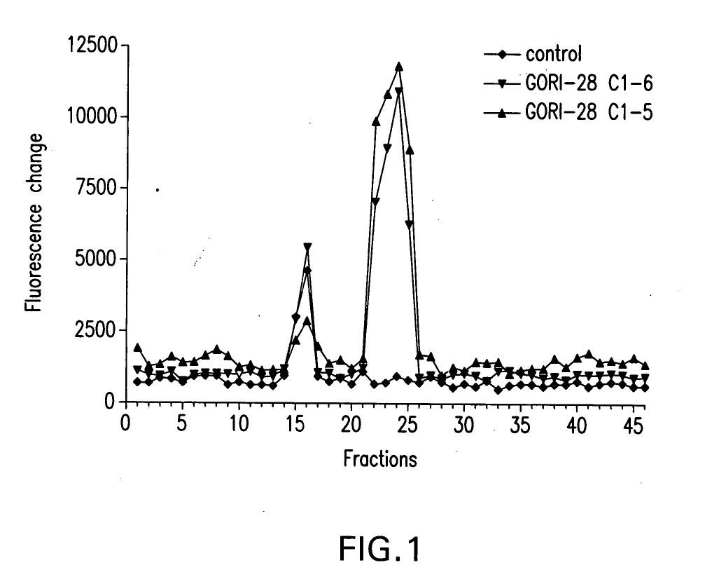

FIG. 1

Specific activation of GORI-28 cells (clones C1-6 and C1-5) by fractions 22-25 of the pH pool 7. The control cells do not express GORI-28. Non-specific activity can be seen in fractions 15-16.

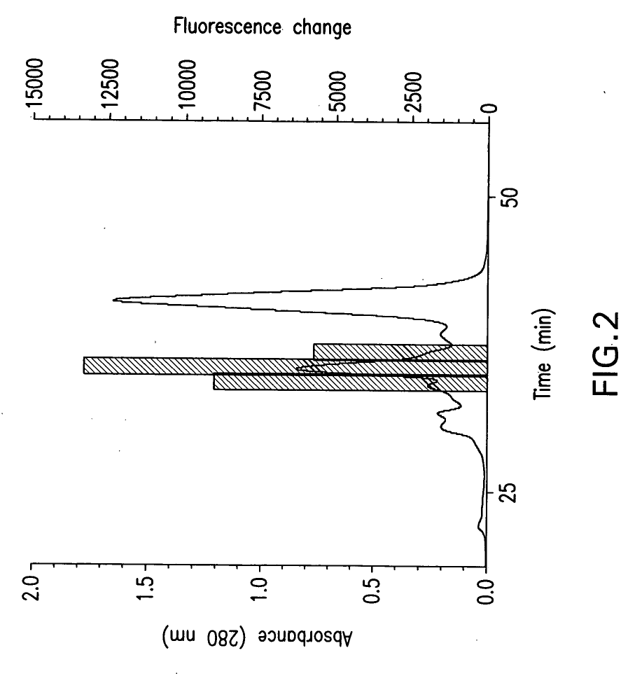

FIG. 2

Step 3 of the chromatographic purification of COM through a Bakerbond RP-C18 column.

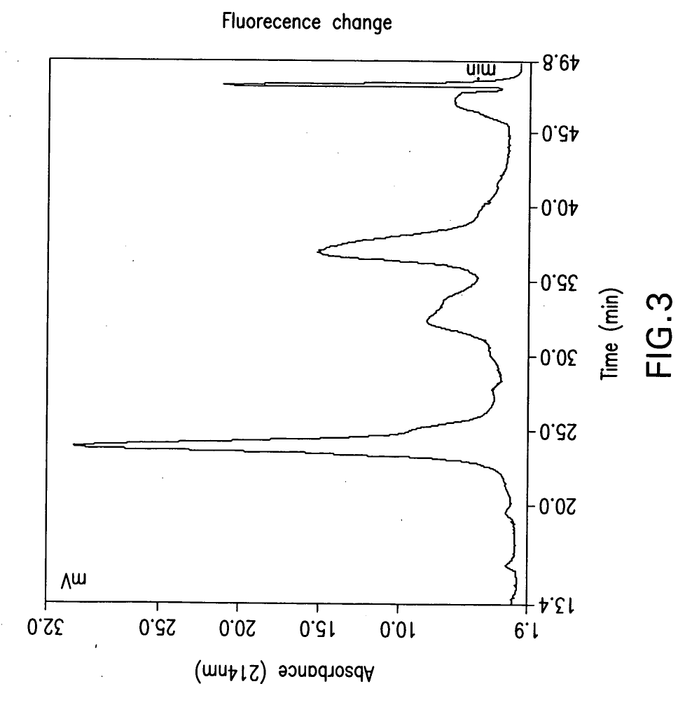

FIG. 3

Step 7 of the chromatographic purification of COM through a Poly Hydroxyethyl HILIC column.

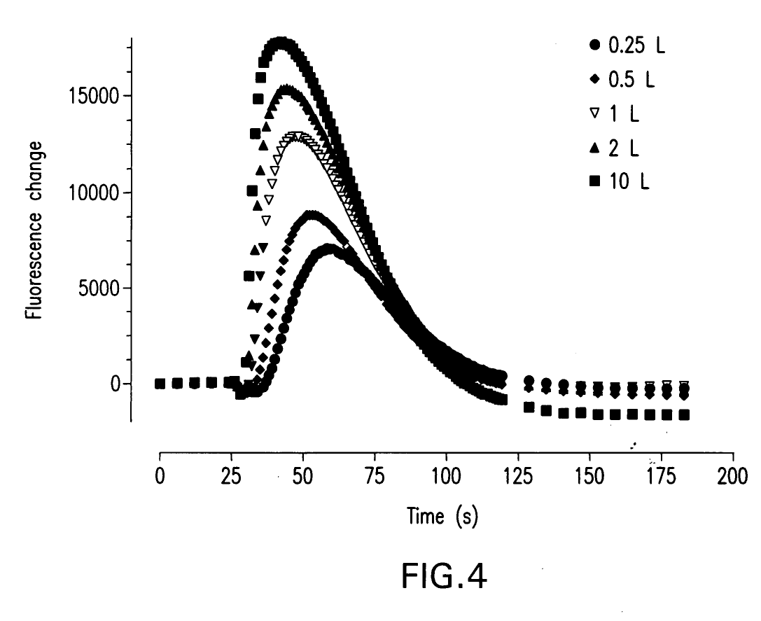

FIG. 4

Dose-effect correlation. The change in fluorescence is plotted against the volume employed of fraction 30 of the 8th purification step (see Example 4).

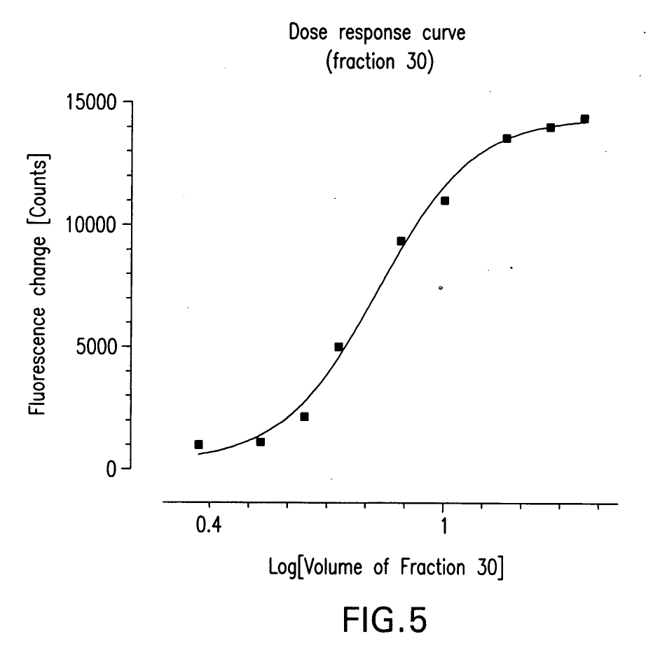

FIG. 5

Dose-effect curve. The change in fluorescence is plotted against the volume employed of fraction 30 of the 8th purification step (see Example 4) on a semi-logarithmic scale.

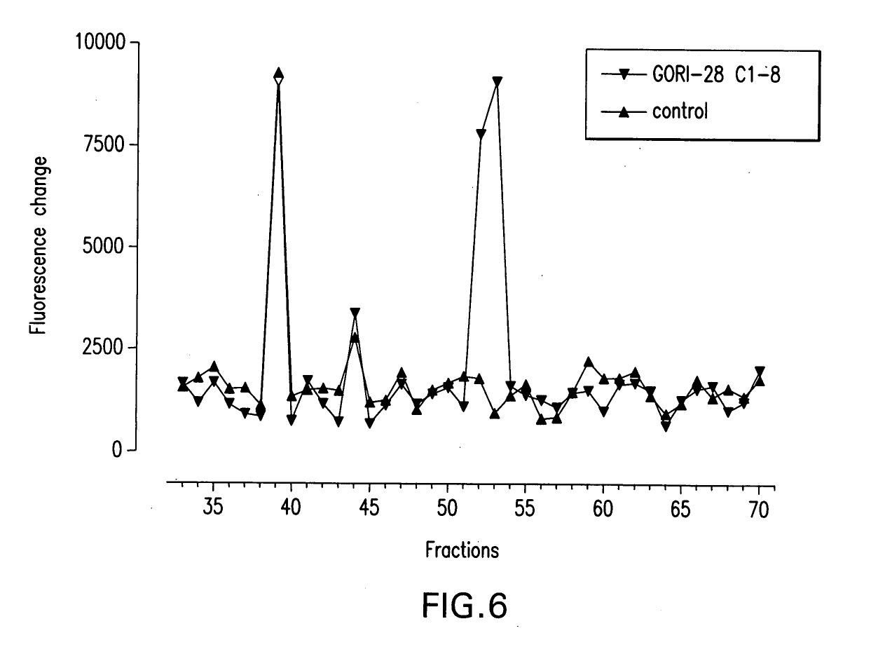

FIG. 6

Activation of GORI-28 cells with recombinant TIG2 from conditioned medium of overexpressing CHO cells purified through a Source RPC15 (10×250 mm).

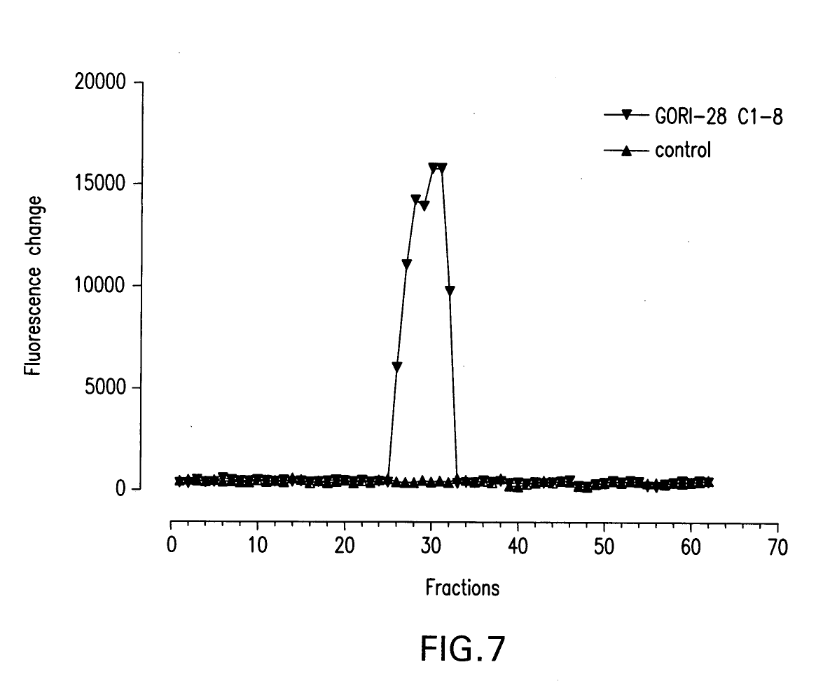

FIG. 7

Activation of GORI-28 cells with recombinant COM purified from cell supernatant of an overexpressing yeast clone.

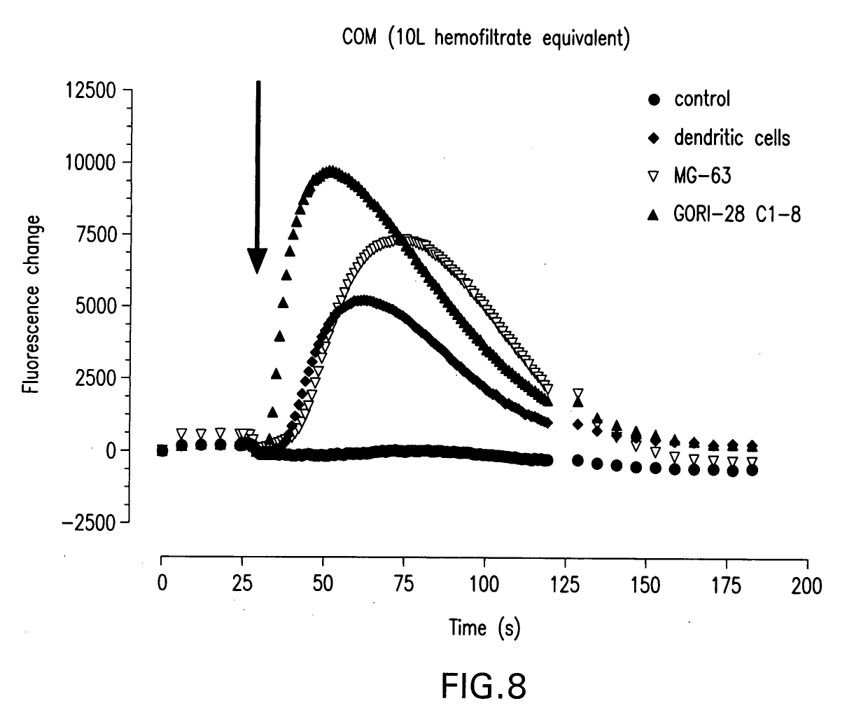

FIG. 8

Activation of osteogenic cells (MG-63) and dendritic cells (DC) with COM purified from hemofiltrate. As positive and negative controls, GORI-28 expressing and non-expressing CHO cells, respectively, are shown.

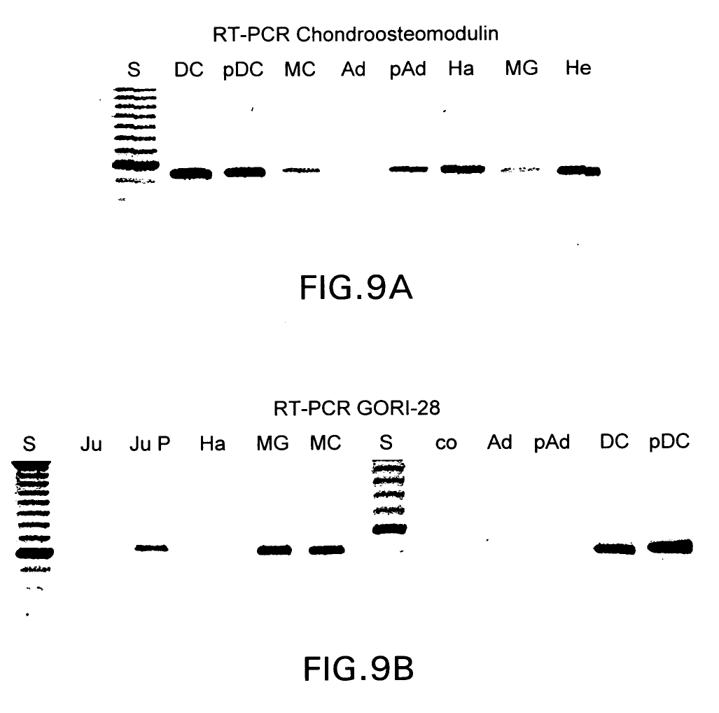

FIG. 9

Expression of GORI-28 and TIG2 in various cell types.

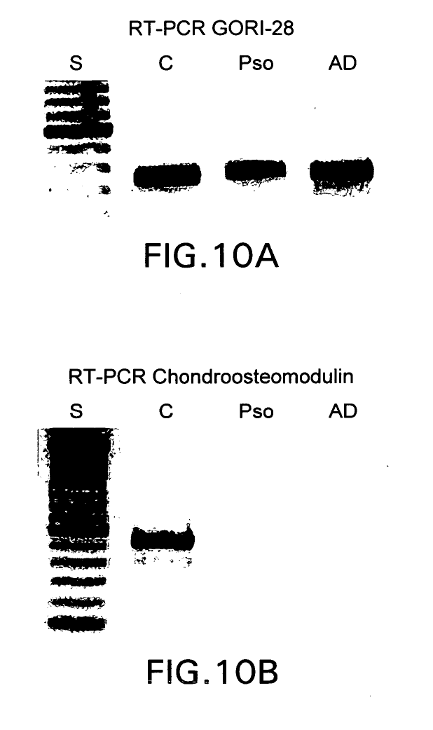

FIG. 10

Expression of GORI-28 and TIG2 in skin samples from patients with skin diseases.

The object of the invention is achieved by COM having the amino acid sequence SEQ ID NO. 1

| | |
|---|---|
| 1 | ELTEAQRRGL QVALEEFHKH PPVQWAFQET SVESAVDTPF PAGIFVRLEF |
| 51 | KLQQTSCRKR DWKKPECKVR PNGRKRKCLA CIKLGSEDKV LGRLVHCPIE |
| 101 | TQVLREAEEH QETQCLRVQR AGEDPHSFYF PGQF |

and its natural and pharmacologically acceptable derivatives, especially amidated, acetylated, phosphorylated and glycosylated derivatives, or having a pyroglutamate at the N terminus.

The invention also relates to derivatives of COM in which the amino acid sequence of COM is changed by amino acid substitutions, insertions or deletions, with the provisos that
the derivatives have a length of not more than 150 amino acids, preferably from 120 to 150, especially from 129 to 140 amino acids;
the derivatives have a sequence identity with COM of more than 80%, preferably more than 90%, especially more than 95%;
the derivatives will activate the receptor GORI-28 in a functional test with the FLIPR system, so that a receptor activity is measured which is at least 50% of the receptor activity triggered by COM under the same testing conditions, preferably being greater than this activity, more preferably exceeding it by at least 20%.

In addition, the object of the invention is achieved by the further embodiments of the invention according to claims 3 to 12.

COM has the capability of influencing the functions of bone cells (osteoblasts and osteoclasts) and cartilage cells as well as fat cells, immunological cells and skin cells. The substance can be obtained from human hemofiltrate (HF) body fluid. The substance is referred to as COM and can be utilized for the purpose of medical and industrial use as a medicament for the treatment or diagnosis of bone and cartilage diseases as well as obesity, diabetes type 2, cancers, tumor metastases, inflammatory diseases, auto-immune diseases, hereditary or acquired immunodeficiencies, tissue rejection, skin diseases, such as psoriasis, eczemas, acne or trophic skin diseases, inflammatory infections, viral, bacterial or parasitic infections, female infertility, ovarian and uterine tumors.

For analyzing substances from peptide libraries, a bioassay has been developed which shows an activation of signal transduction on transfected CHO cells. Surprisingly, in the hemofiltrate, activating substances have been found which influence intracellular Ca²⁺ activation in cell cultures of GORI-28 receptors of transfected cells, also known as ChemR23 (Genbank Accession No. Y14838) or DEZ (Genbank Accession No. U79527). This receptor is functionally active on osteogenic cells, adipocytes, skin cells as well as immunological cells. The influencing was measured, inter alia, on the basis of the stimulation of intracellular Ca²⁺ activation of cells bearing the GORI-28 receptor on their membrane surfaces. Using this test, from an HF peptide library (human blood filtrate), surprisingly, a substance could be identified which shows characteristic elution profiles in the chromatographic purifications. The substance was isolated and identified by relevant mass-spectroscopical analyses as a molecular unit. It is a circulating form of TIG2 (tazarotene-induced responder protein 2; Nagpal S. et al. (1997), J. Invest. Dermatol., 109: 91-95) already found from cloning (Genbank Accession No. U77594).

The cDNA for GORI-28 was cloned from genomic DNA by PCR and subcloned into a eukaryotic expression vector. Stably transfected CHO cell lines which overexpress GORI-28 were produced and subsequently employed in a functional screening assay. Thus, cells expressing GORI-28 were stimulated with fractions from HF, and the receptor activation was followed by the transient increase of the second messenger Ca²⁺. To date, ligands for the receptor GORI-28 have not been described, and therefore, the receptor was classified as an orphan receptor.

The GORI-28 receptor is expressed on developing bone and cartilage cells as well as on dendritic cells, in lymph nodes, spleen, placenta, uterus, lungs, aorta and in the adult parathyroid gland (Samson et al., 1998, Eur. J. Immunol. 28: 1689-1700, Methner et al., 1997, Biochem. Biophys. Res. Commun. 233: 336-342).

TIG2 is expressed in the pancreas, liver, adipocytes, adrenal gland, lung, ovary, uterus, pituitary gland, epidermal cells and osteoclast-supporting stroma cells (Nagpal S. et al., 1997, Adams et al., 1999, J. Cell. Biochem. 74: 587-595). In addition, it was observed that TIG2 is expressed at a reduced level in the damaged tissue of psoriasis patients as compared to non-damaged tissue. After the treatment of damaged tissue with tazarotes, TIG2 expression is induced (Nagpal S. et al., 1997).

From these data and the data stated in the Examples, the physiological significance of COM and GORI-28 in the bone development, energy metabolism, physiology and preservation of the skin and in inflammatory processes.

COM, being an endogenous circulating peptide, is probably more suitable for application as a medicament than other derivatives of the TIG2 gene, because it is not recognized as a foreign substance by the human immune system, and advantageously, an autoimmune response is not to be expected. In addition, the specific processing on both the N and C termini suggests that this is required for the formation of biological function and results in a higher activity.

Thus, the present invention relates to a new osteochondro-active factor, COM, having the following molecular properties:

(1) Molecular weight of precursor (from gene data): 18,617 Da

(2) Molecular weight of found protein COM: 15,566 Da

(3) Chromatographic behavior: see Example 5

(4) Amino acid sequence: according to claim 1

(5) pI value: 8.60

### EXAMPLES

### Example 1

### Cloning of the GORI-28/ChemR23 Receptor

GORI-28 was described by Samson et al. (1998) as ChemR23 and by Methner et al. (1997) as DEZ, and several data base entries exist. The analysis of a genomic clone (Genbank Accession No. NT_{—}009660.4) showed that the described variants of DEZ isoform A (Genbank Accession No. U79526) and isoform B (Genbank Accession No. U79527) are the products of alternative splicings. DEZ isoform A is identical in amino acid sequence with ChemR23 and DEZ isoform B, the N terminus of isoform A being extended by two amino acids, methionine and arginine. To enable simplified cloning from genomic DNA, a genomic clone for ChemR23/DEZ isoform B, which obtained the designation GORI-28, was produced in silico. As compared to the sequence of ChemR23 (Genbank Accession No. Y14838), GORI-28 contains a guanine in position 900, which represents a silent mutation, and from position 1294, it has a sequence which completely differs from the published sequence. The cDNA for GORI-28 was amplified from genomic DNA by PCR using the primers 5′ TGG TCC CTG TCT TCT CTT GC 3′ (GORI 28oli1) and 5′ TGT CCC TGG GTT GAG AGA GT 3′(GORI28oli2) to obtain a 1186 bp fragment which was subsequently subcloned into the expression vector pCI or other usual expression vectors. The sequence was checked by DNA sequence analysis and confirmed.

The GORI-28 cDNA has the following polynucleotide sequence SEQ ID No. 2:

| | |
|---|---|
| 1 | TGGTCCCTGT CTTCTCTTGC AGAGAATGGA GGATGAAGAT TACAACACTT |
| 51 | CCATCAGTTA CGGTGATGAA TACCCTGATT ATTTAGACTC CATTGTGGTT |
| 101 | TTGGAGGACT TATCCCCCTT GGAAGCCAGG GTGACCAGGA TCTTCCTGGT |
| 151 | GGTGGTCTAC AGCATCGTCT GCTTCCTCGG GATTCTGGGC AATGGTCTGG |
| 201 | TGATCATCAT TGCCACCTTC AAGATGAAGA AGACAGTGAA CATGGTCTGG |
| 251 | TTCCTCAACC TGGCAGTGGC AGATTTCCTG TTCAACGTCT TCCTCCCAAT |
| 301 | CCATATCACC TATGCCGCCA TGGACTACCA CTGGGTTTTC GGGACAGCCA |
| 351 | TGTGCAAGAT CAGCAACTTC CTTCTCATCC ACAACATGTT CACCAGCGTC |
| 401 | TTCCTGCTGA CCATCATCAG CTCTGACCGC TGCATCTCTG TGCTCCTCCC |
| 451 | TGTCTGGTCC CAGAACCACC GCAGCGTTCG CCTGGCTTAC ATGGCCTGCA |
| 501 | TGGTCATCTG GGTCCTGGCT TTCTTCTTGA GTTCCCCATC TCTCGTCTTC |
| 551 | CGGGACACAG CCAACCTGCA TGGGAAAATA TCCTGCTTCA ACAACTTCAG |
| 601 | CCTGTCCACA CCTGGGTCTT CCTCGTGGCC CACTCACTCC CAAATGGACC |
| 651 | CTGTGGGGTA TAGCCGGCAC ATGGTGGTGA CTGTCACCCG CTTCCTCTGT |
| 701 | GGCTTCCTGG TCCCAGTCCT CATCATCACA GCTTGCTACC TCACCATCGT |
| 751 | GTGCAAACTG CAGCGCAACC GCCTGGCCAA GACCAAGAAG CCCTTCAAGA |
| 801 | TTATTGTGAC CATCATCATT ACCTTCTTCC TCTGCTGGTG CCCCTACCAC |
| 851 | ACACTCAACC TCCTAGAGCT CCACCACACT GCCATGCCTG GCTCTGTCTT |
| 901 | CAGCCTGGGT TTGCCCCTGG CCACTGCCCT TGCCATTGCC AACAGCTGCA |
| 951 | TGAACCCCAT TCTGTATGTT TTCATGGGTC AGGACTTCAA GAAGTTCAAG |
| 1001 | GTGGCCCTCT TCTCTCGCCT GGTCAATGCT CTAAGTGAAG ATACAGGCCA |
| 1051 | CTCTTCCTAC CCCAGCCATA GAAGCTTTAC CAAGATGTCA TCAATGAATG |
| 1101 | AGAGGACTTC TATGAATGAG AGGGAGACCG GCATGCTTTG ATCCTCACTG |
| 1151 | TGGAACCCCT CAATGGACTC TCTCAACCCA GGGACA |

### Example 2

### Production of CHO Cells which Overexpress GORI-28

The expression vector with the cDNA for GORI-28 was transfected with the transfection reagent Effectene or other usual transfection reagents according to the manufacturer's instructions into CHO cells which endogenously express the G protein α16. Stably transfected cell clones were selected in the presence of neomycin (G-418), and the cell clones obtained were examined for expression of GORI-28 by Northern blot analysis. Cell clones with different levels of expression (GORI-28 C1-5, C1-6, C1-8) were selected for screening with peptide fractions (see Example 3).

### Example 3

### Functional Test for GORI-28

By means of the FLIPR system (Fluorometric Imaging Plate Reader, Molecular Devices), changes of the intracellular calcium concentration can be detected. After stimulating the receptor, the intracellular messenger IP3 is released which opens IP3-specific channels of the endoplasmic reticulum and causes Ca²⁺ ions to flow into the cytosol. Before the measurement, the cells are loaded with the calcium-sensitive dye Fluo-4 (Molecular Probes). Ca²⁺ ions will bind to Fluo-4, and after exciting the Fluo-4/Ca²⁺ complex by an argon laser (488 nm), the emission is measured at a wavelength of 540 nm. This light signal is detected and recorded by a CCD camera and subsequently evaluated with a computer program. Cells are sown in 96-well plates at 20 000 cells/well and incubated over night. The following day, the cells were loaded with 2 μM Fluo-4 AM for 40 min in hepes/HBSS buffer, pH 7.4, 2.5 mM probenecid, then washed and incubated with 100 μl of hepes/HBSS, pH 7.4, 2.5 mM probenecid for 5 min. After the addition of 50 μl of hemofiltrate fraction or other test substrates, the changes of intracellular fluorescence are recorded on-line.

### Example 4

### Isolation of HF-chondroosteomodulin

The isolation of COM from 8000 liters of human hemofiltrate was effected according to the following Example:

Isolation of HF-chondroosteomodulin from hemofiltrate:
Purification strategy
Collection of 8000 I batch of human hemofiltrate

| | |
|---|---|
| Cation exchange chromatography (step elution) | 1st step |
| RP-Fineline Source C15 chromatography (gradient elution) | 2nd step |
| Bakerbond RP C18 column chromatography (gradient elution) | 3rd step |
| RP-Biotek C4 column chromatography (gradient elution) | 4th step |
| RP-Vydac C18 column chromatography (gradient elution) | 5th step |
| RP-Phenomenex C5 column chromatography (gradient elution) | 6th step |
| Poly Hydroxyethyl HILIC column chromatography (gradient | 7th step |
| elution) | |
| RP-Phenomenex C5 column chromatography (gradient elution) | 8th step |
| High purity achieved | |

8000 liters of hemofiltrate was subjected to ultrafiltration through a membrane with a cut-off size of 50 kD, then bound to a cation-exchange column (Fractogel SP 650 (M)), then eluted stepwise with 7 buffers with increasing pH value (pH pools 1-7, 7 eluates). In a second step, each eluate was subjected to chromatography through a Fineline Source RP-C (C15, 10×15.5 cm, 300 Å), so that about 46 fractions were obtained for each pH pool. For the biotest, lyophilized aliquots were reconstituted in hepes/HBSS buffer and used as described in Example 3. Fractions 22-25 of pH pool 7 exhibited a specific activity on GORI-28 cells, but not on a control cell line which does not express the receptor (see FIG. 1). About 700 mg of lyophilized mother fractions of the GORI-28-activating fractions were combined and purified in six further steps (see Scheme). The COM separated to high purity exhibits the chromatographic, mass-spectrometric and molecular properties as shown in Example 5.

### Example 5

### Chromatographic, Mass-Spectrometric and Molecular Properties

From the chromatographic purification of COM, steps 3 and 7 are shown in FIGS. 1 and 2 by way of example; the fractions in which the biological activity was found are labeled.

FIG. 4 shows a dose-effect correlation of COM. The change in fluorescence is plotted against the volume employed of fraction 30 of the 8th purification step (see Example 4).

FIG. 5 shows a dose-effect curve of COM. The change in fluorescence is plotted against the volume employed of fraction 30 of the 8th purification step (see Example 4).

The purity of COM was checked by capillary zone electrophoresis (P/ACE 2000, Beckman) (not shown). The determination of the molecular mass was effected by a Voyager DE PRO mass spectrometer (PerSpective), and a mass of 15,562 Da was established. The N terminus and the first 33 amino acids were determined by Edman degradation (Applied Biosystems Gas Phase Sequencer 473 A). From these data, the amino acid sequence of COM with 134 amino acids and a theoretical molecular weight of 15,566 Da can be derived, taking into account that the six cysteine residues form three disulfide bridges. The amino acid sequence determined of COM reads:

| | |
|---|---|
| 1 | ELTEAQRRGL QVALEEFHKH PPVQWAFQET SVESAVDTPF PAGIFVRLEF |
| 51 | KLQQTSCRKR DWKKPECKVR PNGRKRKCLA CIKLGSEDKV LGRLVHCPIE |
| 101 | TQVLREAEEH QETQCLRVQR AGEDPHSFYF PGQF |

### Example 6

### Cloning and Recombinant Expression of TIG2 in CHO Cells

The cDNA for human TIG2 (Genbank Accession No. U77594) was amplified from liver cDNA by PCR with the primers 5′ GCCAGGGTGACACGGAAG 3′ (TIG2oli1) and 5′ GAGGCACCACGCAGCTC 3′ (TIG2oli2) to obtain a fragment of 537 bp, which was subcloned into the vector pGEM5Zf-T or other usual vectors. The sequence was checked by DNA sequence analysis and confirmed. From this recombinant vector, a fragment which contains the cDNA of TIG2 was excised with suitable restriction enzymes and subcloned into the expression vector pCI or other usual expression vectors. CHO cells were transfected with the recombinant expression vector as in Example 2, and stable cell clones were selected as in Example 2. The cell clones obtained were examined for the expression of TIG2 by RT-PCR.

### Example 7

### Activation of GORI-28 with Recombinant TIG2

A TIG2-expressing cell clone was expanded for the production of TIG2. Four confluent 75 cm jars were washed twice with PBS, and subsequently medium without FCS was applied. After 72 h of incubation, the conditioned medium was taken off, subjected to centrifugation at 500 g for 5 min to remove cell debris, and then purified through a Source RPC15 (10×250 mm). The fractions obtained were tested in a FLIPR assay (see Example 3) for GORI-28-stimulating activity. Activity was found in fractions 52 and 53 which stimulate GORI-28 cells, but not a control cell line (see FIG. 6). For control, conditioned medium of CHO cells which express another peptide was used. In this conditioned medium, no GORI-28-stimulating activity was found (not shown).

### Example 8

### Cloning and Recombinant Expression of COM in Yeast

By PCR with the primers 5′CTCTCGAGAAAAGAGAGCTCACGGAAGCCCAGC 3′ (COMoli1) and 5′ TTGTCGACTTAGMCTGTCCAGGGAAGTAGAAGC 3′ (COMoli2) and using the TIG2 cDNA cloned in Example 6, a 427 bp fragment which represents the cDNA for COM was amplified and subsequently subcloned into the vector pGEM5Zf-T or other usual vectors. After confirming the sequence by DNA sequence analysis, a fragment was excised from the recombinant vector by means of suitable restriction enzymes and subcloned into the modified yeast expression vector YEpFLAG-1 or other usual expression vectors. The yeast strain BJ3505 or other usual yeast strains were transformed with the expression construct by electroporation. The thus formed ADH2+ clones were checked by PCR analysis for insertion of the COM DNA into the yeast genome. For the preparation of recombinant COM, 10 ml cultures were inoculated with COM-positive clones, and expression was induced. After 96 h, the cell supernatants were harvested and tested for expression of the recombinant COM after separation through a gel (SDS PAGE) and staining with Coomassie blue. In the yeast expression system described, the cDNA of COM is fused to the N-terminal signal of the yeast alpha factor. The alpha factor signal sequence causes the fusion product to be secreted into the cell medium. The alpha factor signal sequence is cleaved off by the endogenous protease Kex 2 to form mature COM.

### Example 9

### Purification of Recombinant COM and Detection of its Activity

Recombinant COM was purified from the cell supernatant of a COM-expressing yeast clone. The cell supernatant was filtered (0.2 μM filter), diluted three times with buffer A (10 mM Na₂HPO₄, pH 7.0), applied to a heparin column (Hightrap), and eluted with buffer B (buffer A with 1.5 M NaCl). The fractions obtained were tested in a FLIPR assay (see Example 3) for the localization of the GORI-28-stimulating activity. In the second purification step, the active fractions were combined, applied to an RPC15 column and eluted. As previously, the active fractions were determined by the functional assay, combined and purified through a Phenomenex C18 column in the third purification step. Purified recombinant COM shows specific activity on GORI-28 cells, but not on a control cell line which does not express the receptor (see FIG. 7).

### Example 10

### Activation of Osteogenic Cells by COM

For examining the functional activation of bone cells by COM, MG-63 cells, an established human osteosarcoma cell line (osteoblast-like type), were sown in 96-well plates at 20,000 cells per well on the previous day and tested in a FLIPR assay as described in Example 3. The cells were stimulated with COM purified from HF (see Examples 4 and 5) and showed a COM-induced release of Ca²⁺ ions (see FIG. 8). As positive and negative controls, the cell line which overexpresses GORI-28 and a CHO cell line which expresses another G protein-coupled receptor are shown.

### Example 11

### Activation of Dendritic Cells by COM

Dendritic cells were obtained from human whole blood. At first, monocytes (CD14+) were isolated from whole blood (500 ml) through several centrifugation steps and separation by means of paramagnetic antibodies (anti-CD14). The precursor cells obtained were treated with GM-CSF (800 U/ml) and interleukin-4 (500 U/ml) to induce differentiation into dendritic cells (incubation period: 7 days). Subsequently, the cells were stimulated with LPS to obtain so-called mature dendritic cells. These mature dendritic cells were sown in 96-well plates at a cell density of 20,000 cells per well on the previous day, followed by testing in an FLIPR assay as described in Example 3. The cells were stimulated with COM purified from HF (see Examples 4 and 5) and showed a COM-induced release of Ca 2+ ions (see FIG. 8).

### Example 12

### Expression of TIG2 and GORI-28 in Selected Cell Lines

The native expression of TIG2 and GORI-28 in various cell lines was examined by RT-PCR with gene-specific primers. COM was amplified with the TIG2oli1 and TIG2oli2 pair of primers (see Example 6) as a 537 bp cDNA fragment and isolated by gel electrophoresis. By analogy, GORI-28 was amplified and detected by means of the primer pair 5′ GGC CAT GTG CM GAT CAG CAA CT 3′ (mDEZoli1) and 5′ AGA ATG GGG TTC ATG CAG CTG TT 3′ (mDEZoli2) as a 618 bp fragment; for the PCR amplification from murine adipocytes, the primer pair 5′ TCT ACA ACG GTG GAA CAG TGA 3′ (mDEZoli3) and 5′ AAG AAA GCC AGG ACC CAG A 3′ (mDEZoli4) was employed to form a 536 bp fragment; for the amplification from human dendritic cells, the primer pair 5′ CAG ACA ACA TAA CGG TGA ATG A 3′ (hDEZ_a_Oli5) and 5′ AAG AAA GCC AGG ACC CAG A 3′ (hDEZ_a_Oli4) was employed to form a 524 bp fragment. From cell samples of the cells to be examined, RNA was isolated by the usual methods, then transcribed into a first strand cDNA which was employed for PCR amplification.

The expression of the ligand COM could be detected in mature human dendritic cells (DC) and precursor cells (pDC), murine osteosarcoma cells MC3T3 (osteoblast-like type, MC), mature murine adipocytes (fat cells, Ad) and precursor adipocytes (pAd), human ceratinocytes (HaCaT, Ha), human osteosarcoma cells MG-63 (osteoblast-like type, MG), and human hepatocytes HepG2 (He) (see FIG. 9A).

The expression of the COM receptor GORI-28 was detected in Jurkat T cells (human leukemic T cell line, Ju), at an enhanced level in PMA/ionomycin-activated Jurkat T cells (Ju P), in HaCaT cells (Ha), MG-63 cells (MG), MC3T3 (MC), dendritic (DC) and precursor cells (pDC), in mature adipocytes (Ad), but not in precursor cells (pAd) (see FIG. 9B). In FIG. 9B, a negative control (co) is shown in which the cDNA as a template was replaced by a water sample.

The expression analysis of the COM receptor GORI-28 shows that its expression is controlled by physiological processes. In the example shown, the expression of the receptor could be increased by in vitro T cell activation and induced in immature adipocytes by differentiation. In Jurkat T cells, T cell activation was achieved by means of phorbol ester and ionomycin. The differentiation of the immature adipocytes was induced by dexamethasone, 8BrcAMP and insulin.

### Example 13

### Expression of COM and GORI-28 in Skin Cells from Patients with Skin Diseases

As described in Example 12, the expression of COM and its receptor GORI-28 was established by RT-PCR. For the amplification of GORI-28, in this case, the primer pair 5′ GCA CAG CAT CAC TTC TAC CAC TT 3′ (hDEZoli3) and 5′ CTG TAG ACC ACC ACC AGG AAG A 3′ (hDEZoli2) was used to form a 324 bp fragment. Skin punches from patients with no skin disease (control, C) and from patients suffering from psoriasis (Pso) or atopic dermatitis (AD) were obtained from a skin hospital. The material was prepared by the usual methods to isolate RNA, from which a first strand cDNA was in turn synthesized as described in Example 12 and employed for PCR amplification. The receptor GORI-28 is expressed both in the skin tissue of healthy subjects and in the skin tissue of patients with psoriasis or atopic dermatitis (see FIG. 10 A). In contrast, COM expression could be detected only in the tissue of healthy subjects, but not in the skin tissue of patients who suffer from psoriasis or atopic dermatitis (see FIG. 10 B). Thus, the lack of COM expression is in a causal relationship with skin diseases and indicates a therapeutic effectiveness of COM for this field of indications.

To conclude, it could be shown that the circulating substance COM occurs in human hemofiltrate and could be isolated and characterized. It exerts osteochondro-anabolic, immunomodulatory activities and activities regulating skin and energy metabolism.

1-10. (canceled)

11. A HF-chondroosteomodulin (COM) polypeptide or derivative thereof having the amino acid sequence SEQ ID NO. 1

| | |
|---|---|
| 1 | ELTEAQRRGL QVALEEFHKH PPVQWAFQET SVESAVDTPF PAGIFVRLEF |
| 51 | KLQQTSCRKR DWKKPECKVR PNGRKRKCLA CIKLGSEDKV LGRLVHCPIE |
| 101 | TQVLREAEEH QETQCLRVQR AGEDPHSFYF PGQF |

provided that,
the derivatives have a length of not more than 150 amino acids;
the derivatives have a sequence identity with COM of more than 80%;
the derivatives will activate the receptor GORI-28 in a functional test with the FLIPR system, so that a receptor activity is measured which is at least 80% of the receptor activity triggered by COM under the same conditions.

12. The COM polypeptide or derivative of claim 11, selected from the group consisting of: amidated, acetylated, phosphorylated and glycosylated derivatives; or having a pyroglutamate at the N terminus, in which the amino acid sequence of the derivatives is changed by amino acid substitutions, insertions or deletions.

13. The COM polypeptide or derivative thereof of claim 11, further comprising a GORI-28 receptor.

14. A method of screening a library with a COM polypeptide or derivative thereof and a GORI-28 receptor as a ligand-receptor system comprising, contacting peptide libraries or other substance libraries with said ligand-receptor system.

15. A method of preparing the COM polypeptide or derivative thereof of claim 11, comprising:
a) preparing said polypeptide or derivative thereof by cell cultures and purifying by chromatography;
b) isolating said polypeptide or derivative thereof from human blood by chromatography; or,
c) preparing said polypeptide or derivative thereof by chemical or biotechnological synthesis, and purifying by chromatography.

16. A pharmaceutical composition comprising the COM polypeptide or derivative thereof of claim 11, optionally in addition to usual adjuvants and additives.

17. The pharmaceutical composition of claim 16, wherein said composition is a lyophilized form taken up with mannitol.

18. The pharmaceutical composition of claim 17, comprising a galenic dosage form containing an amount of from 300 μg to 30 mg of purified COM per therapy unit in sterile ampoules for dissolution in physiological saline and/or infusion solutions for repeated single injection and/or permanent infusion.

19. A method of treating a disease in a human comprising, administering an effective amount of the COM polypeptide or derivative thereof of claim 16, wherein said disease is selected from the group consisting of: diseases of the parathyroid gland; degenerative bone diseases; bone fractures in the phase of healing; cartilage diseases; connective tissue diseases; rheumatism; arthrosis; obesity; diabetes type 2; diseases of the immune system; diseases subjected to therapy influencing the migration of stem cells, including chemotherapy; renal diseases accompanied by disorders in electrolyte excretion, and skin diseases.

20. The method of claim 19, wherein said disease is selected from the group consisting of: hypoparathyroidism, osteoporosis, acute renal insufficiency, phosphate and calcium excretion disorders, psoriasis, eczema and acne.

21. A method of diagnosing a disease in a human with the COM polypeptide or derivative thereof of any one of claims 11 and 16 comprising:
a) preparing specific antibodies against said polypeptide or derivative thereof;
b) contacting said antibodies with a tissue sample from said human; and,
c) measuring the concentration of said COM polypeptide or derivative thereof by immune tests or by quantitative mass spectrometry.

22. The method of claim 21, wherein said pharmaceutical composition is in the form of a galenic dosage.

23. The method of claim 22, wherein said pharmaceutical composition is a lyophilizate galenic dosage.

24. The method of claim 23, wherein said galenic dosage form, comprises an amount of from 300 μg to 30 mg of pureified COM polypeptide or derivative thereof per therapy unit in sterile ampoules for dissolution in physiological saline and/or infusion solutions for the repeated single injection and/or permanent infusion.

25. The COM polypeptide or derivative of claim 11, wherein said receptor activity triggered by the COM polypeptode or derivative thereof is greater than the receptor activity triggered by COM.

26. An immunoassay kit comprising, specific antibodies against said polypeptide or derivative thereof of any one of claims 11 and 16.


SEQUENCE LISTING (16 sequences)

SEQ ID NO: 1 - protein, 134 aa, Homo sapiens
ELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQF

SEQ ID NO: 2 - DNA, 1186 bp, Artificial Sequence
  Description of artificial sequence  GORI-28 cDNA
tggtccctgtcttctcttgcagagaatggaggatgaagattacaacacttccatcagttacggtgatgaataccctgattatttagactccattgtggttttggaggacttatcccccttggaagccagggtgaccaggatcttcctggtggtggtctacagcatcgtctgcttcctcgggattctgggcaatggtctggtgatcatcattgccaccttcaagatgaagaagacagtgaacatggtctggttcctcaacctggcagtggcagatttcctgttcaacgtcttcctcccaatccatatcacctatgccgccatggactaccactgggttttcgggacagccatgtgcaagatcagcaacttccttctcatccacaacatgttcaccagcgtcttcctgctgaccatcatcagctctgaccgctgcatctctgtgctcctccctgtctggtcccagaaccaccgcagcgttcgcctggcttacatggcctgcatggtcatctgggtcctggctttcttcttgagttccccatctctcgtcttccgggacacagccaacctgcatgggaaaatatcctgcttcaacaacttcagcctgtccacacctgggtcttcctcgtggcccactcactcccaaatggaccctgtggggtatagccggcacatggtggtgactgtcacccgcttcctctgtggcttcctggtcccagtcctcatcatcacagcttgctacctcaccatcgtgtgcaaactgcagcgcaaccgcctggccaagaccaagaagcccttcaagattattgtgaccatcatcattaccttcttcctctgctggtgcccctaccacacactcaacctcctagagctccaccacactgccatgcctggctctgtcttcagcctgggtttgcccctggccactgcccttgccattgccaacagctgcatgaaccccattctgtatgttttcatgggtcaggacttcaagaagttcaaggtggccctcttctctcgcctggtcaatgctctaagtgaagatacaggccactcttcctaccccagccatagaagctttaccaagatgtcatcaatgaatgagaggacttctatgaatgagagggagaccggcatgctttgatcctcactgtggaacccctcaatggactctctcaacccagggaca

SEQ ID NO: 3 - DNA, 18 bp, Artificial Sequence
  Description of artificial sequence  Primer TIG2oli1
gccagggtgacacggaag

SEQ ID NO: 4 - DNA, 17 bp, Artificial Sequence
  Description of artificial sequence  Primer TIG2oli2
gaggcaccacgcagctc

SEQ ID NO: 5 - DNA, 33 bp, Artificial Sequence
  Description of artificial sequence  Primer COMoli1
ctctcgagaaaagagagctcacggaagcccagc

SEQ ID NO: 6 - DNA, 34 bp, Artificial Sequence
  Description of artificial sequence  Primer COMoli2
ttgtcgacttagaactgtccagggaagtagaagc

SEQ ID NO: 7 - DNA, 23 bp, Artificial Sequence
  Description of artificial sequence  Primer mDEZoli1
ggccatgtgcaagatcagcaact

SEQ ID NO: 8 - DNA, 23 bp, Artificial Sequence
  Description of artificial sequence  Primer mDEZoli2
agaatggggttcatgcagctgtt

SEQ ID NO: 9 - DNA, 21 bp, Artificial Sequence
  Description of artificial sequence  Primer mDEZoli3
tctacaacggtggaacagtga

SEQ ID NO: 10 - DNA, 19 bp, Artificial Sequence
  Description of artificial sequence  Primer mDEZoli4
aagaaagccaggacccaga

SEQ ID NO: 11 - DNA, 22 bp, Artificial Sequence
  Description of artificial sequence  Primer hDEZ_a_Oli5
cagacaacataacggtgaatga

SEQ ID NO: 12 - DNA, 19 bp, Artificial Sequence
  Description of artificial sequence  Primer hDEZ_a_Oli4
aagaaagccaggacccaga

SEQ ID NO: 13 - DNA, 23 bp, Artificial Sequence
  Description of artificial sequence  Primer hDEZoli3
gcacagcatcacttctaccactt

SEQ ID NO: 14 - DNA, 22 bp, Artificial Sequence
  Description of artificial sequence  Primer hDEZoli2
ctgtagaccaccaccaggaaga

SEQ ID NO: 15 - DNA, 20 bp, Artificial Sequence
  Description of artificial sequence  Primer GORI28oli1
tggtccctgtcttctcttgc

SEQ ID NO: 16 - DNA, 20 bp, Artificial Sequence
  Description of artificial sequence  Primer GORI28oli2
tgtccctgggttgagagagt